(19) United States
(12) Patent Application Publication
(10) Pub. No.: US 2015/0017711 A1
(43) Pub. Date: Jan. 15, 2015

(54) CELL CULTURE SYSTEMS

(71) Applicant: CORNING INCORPORATED, CORNING, NY (US)

(72) Inventors: Scott Matthew Bennett, Gray, ME (US); David Alan Kenney, Chelmsford, MA (US); Gregory Roger Martin, Acton, ME (US); Allison Jean Tanner, Portsmouth, NH (US)

(21) Appl. No.: 14/375,553

(22) PCT Filed: Jan. 31, 2013
(86) PCT No.: PCT/US13/23981

§ 371 Date: Jul. 30, 2014

Related U.S. Application Data
(60) Provisional application No. 61/594,034, filed on Feb. 2, 2012.

(51) Int. Cl. C12M 3/06; C12M 1/26; C12M 1/34; C12M 1/00; C12M 1/36

(52) U.S. Cl. CPC C12M 27/16; C12M 29/14; C12M 41/48; C12M 23/50; C12M 41/40; C12M 41/44; C12M 33/08
USPC 435/286.2; 435/303.3; 435/286.5; 435/286.1

(57) ABSTRACT

An integrated cell culture system may include one or more cell culture vessels, manipulation apparatus, pumping apparatus, cell release apparatus, monitoring apparatus, and a control apparatus. The control apparatus may be used to monitor and control the system to facilitate effective cell culturing. The cell release apparatus may be used to release a plurality of cells adhered to the cell culture surfaces of the cell culture vessels.

This application claims the benefit of priority under 35 U.S.C. §119 of U.S. Provisional Application Ser. No. 61/594,034 filed on Feb. 2, 2012 the content of which is relied upon and incorporated herein by reference in its entirety.

### FIELD

The present disclosure relates to systems configured to perform cell culturing.

### BACKGROUND

Many types of closed system cell culture articles for high yield cell growth are available such as, e.g., Corning's CELLSTACK cell culture vessel, Corning's HYPERSTACK cell culture vessel, etc. The HYPERSTACK cell culture vessel includes a multi-layered design that utilizes Corning's gas-permeable-film technology.

Often, laboratories performing static adherent, or anchorage dependent, cell culture use large numbers of disposable cell culture articles. To aid in processing the large numbers of cell culture vessels, automated vessel handling equipment has been developed, such as TAP BIOSYSTEMS SELECT and COMPACT systems, which are room-sized cell culture systems that utilize a robotic arm to perform manipulations for the cell cultures. While these systems have the capacity to shake vessels during the course of traditional enzymatic cell removal/release, they do not have any method for removing cells by inducing a vibrational wave front that causes the cells to dissociate from the culture surface of vessels being manipulated. For such systems, cells are typically removed by enzymatic methods, which may not be preferred by regulatory agencies because enzymes may damage the cells in culture and may have a risk of contamination from animal derived components.

While some automated vessel handling systems can process cell culture vessels by piercing septa located in a cap of the vessel, such as the TECAN CELLERITY, such systems are not true closed systems. Regulatory agencies concerned with therapeutic use of cells, or agents produced by cells, in culture may also prefer all cell culture-derived therapeutic product manufacturing to be conducted using “closed systems.” Vessels with caps and septa are not true “closed systems.”

A commercially available manipulator for use with stacked cell culture vessels is available from Nunc—the AUTOMATIC CELL FACTORY MANIPULATOR (ACFM). The AFCM lifts and rotates stacked cell culture vessels when a human operator presses a button. Other operations must be carried out by a human operator such as, e.g., adding or removing various liquids used during the cell culture processes. Further, the AFCM does not conduct in-line monitoring of processes, the cells in culture, or vessel integrity. Further, the dimensions (approximately 4 feet by 6 feet) of the AFCM are such that it cannot pass through a standard door opening (e.g., 3 feet wide). Once installed, the AFCM may be very difficult to move. A separate incubator is available for the AFCM, but, like the AFCM, the incubator does not fit through a standard door opening (e.g., 3 feet wide).

While some bioreactors conduct in-line process monitoring of the cells in culture, such bioreactors are not static cultures, and most bioreactors are for cells in suspension. Further, the bioreactors are often perfused or must have some sort of dynamic movement to keep the cultures suspended.

### BRIEF SUMMARY

The present disclosure describes, among other things, cell culture systems that may provide closed and automatic systems that perform culturing of anchorage dependent, or adherent, cells using one or more cell culture vessels. The cell culture vessels may include one or more cell culture surfaces and at least one port for allowing materials to flow in and out of the cell culture vessel. In embodiments, the system is configured to automatically fill the one or more cell culture vessels with cell culture medium, to release the cells cultured within the one or more cell culture vessels from the one or more cell culture surfaces, and to empty (e.g., harvest) the cultured cells from the one or more cell culture vessels. In embodiments, the system is a closed system, which means that the cell culture vessels are not opened to the outside environment during culturing processes.

In embodiments, cell culture systems enable non-enzymatic cell removal and promote closed system operation, which, e.g., may assist cell-based therapeutic manufacturers to comply with certain regulatory guidelines. Further, the cell culture systems may include manipulation apparatus configured to handle stacked cell culture vessels that have a streamlined design. In embodiments, cell culture systems facilitate totally, or partially, automated cell culture processing that may reduce the possibility of human error.

In various embodiments, cell culture systems incorporate cell release apparatus into the automated cell culture handling/processing apparatus or equipment. Further, cell culture systems may also provide methods for “closed system” processing of cell culture vessels and designs that facilitates equipment portability.

In various embodiments, the present disclosure describes a cell culture system. The cell culture apparatus includes at least one cell culture vessel, manipulation apparatus, pumping apparatus, monitoring apparatus, and control apparatus. The at least one cell culture vessel is configured to culture cells using a plurality of parallel cell culture surfaces and the at least one cell culture vessel includes at least one port configured to allow material to flow into and out of the at least one cell culture vessel. The manipulation apparatus is configured to rotate the at least one cell culture vessel about a first rotation axis and about a second rotation axis (e.g., where the first rotation axis is perpendicular to the second rotation axis, and where each of the first rotation axis and the second rotation axis are parallel a ground surface). The pumping apparatus is fluidly coupled to the at least one port of the at least one cell culture vessel and is configured to pump material into and out of the at least one cell culture vessel through the at least one port. The monitoring apparatus is configured to monitor one or more parameters of the at least one cell culture vessel, the manipulation apparatus, and the pumping apparatus. The control apparatus is operably coupled to the manipulation apparatus, the pumping apparatus, and the monitoring apparatus, and is configured to coordinate movement of the at least one cell culture vessel using the manipulation apparatus with the pumping of material into and out of the at least one culture vessel using the pumping apparatus.

In various embodiments, the cell culture system further includes cell release apparatus configured to release cells adhered to the plurality of parallel cell culture surfaces of the at least one cell culture vessel and the control apparatus is also operably coupled to the cell release apparatus. The control apparatus is configured to execute a cell release process using the cell release apparatus to release at least a portion of a plurality of cells adhered to the plurality of parallel cell culture surfaces of the at least one cell culture vessel.

In various embodiments, the control apparatus is further configured to monitor, using the monitoring apparatus, one or more parameters of the at least one cell culture vessel, the manipulation apparatus, and the pumping apparatus, and adjust one or more parameters of the at least one cell culture vessel, the manipulation apparatus, and the pumping apparatus based on the monitored one or more parameters.

Embodiments of cell culture systems described herein may be configured to fill and empty one or more cell culture vessels autonomously using various apparatus. This and other advantages will be readily understood from the following detailed descriptions when read in conjunction with the accompanying drawings.

### BRIEF DESCRIPTION OF THE DRAWINGS

FIG. 1 is a diagram of a cell culture system.

FIG. 2A is a front view of a cell culture system including, among other things, manipulation apparatus.

FIG. 2B is a side view of the cell culture system of FIG. 2A.

FIG. 2C is another side view of the cell culture system of FIG. 2A with the outrigger portions extended in a deployed position.

FIG. 3A is a front view of the cell culture system of FIG. 2A with the cell culture vessel manipulated into a filling position.

FIG. 3B is a side view of the cell culture system of FIG. 2A with the cell culture vessel manipulated into a filling position.

FIGS. 4A-4C depict another cell culture system with outriggers positioned in a stowed position and in a deployed position, respectively.

FIG. 5 is a material transfer method, e.g., using the cell culture system of FIG. 1.

FIG. 6 is a cell culture vessel integrity check method, e.g., using the cell culture system of FIG. 1.

FIG. 7 is a cell culturing and monitoring method, e.g., using the cell culture system of FIG. 1.

FIG. 8 is a manual cell culture vessel manipulator.

FIG. 9 is another manual cell culture vessel manipulator.

The drawings are not necessarily to scale. Like numbers used in the figures refer to like components, processes and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of apparatus, systems, and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open ended sense, and generally mean “including, but not limited to.”

The present disclosure describes, among other things, cell culture systems that provide automated and closed system cell culture processing using one or more cell culture vessels. Generally, the cell culture systems described herein may provide a fully automated solution to accomplish one or more processes used to seed, grow and harvest adherent cells from stacked cell culture vessels. Further, the systems described herein may bring together multiple discrete components and integrates them with one central computer controlled machine that may use one or more sensing devices to provide feedback to the cell culturist. Further, one or more cell culture systems described herein may include a Human Machine Interface (HMI) that allows users to enter numeric process variables that are specific to their cell culture needs, full computer or Programmable Logic Control (PLC) one or more cell culturing parameters such as, e.g., the fill rate, fill pressure and fill volume of each stacked cell culture vessel, fully automated positioning of vessels coordinated with pump speed adjustment during filling/emptying, equilibration, and cell removal phases, fully automated valves to control the flow of media into and out of the vessel, proper positioning of vent filters to avoid wetting, automated pressure testing to ensure vessel integrity, integrated safety features, process monitoring of time, temperature, pH, gas concentrations and metabolites, enhanced portability through the incorporation of outriggers, and integration of cell release apparatus for enzyme free cell removal. Embodiments of cell culture systems described herein may include various apparatus to provide a streamlined design. Such streamlined design may enable portability of one or more portions (e.g., all) of the system without major disassembly or deconstruction of facility doors and walls. For example, embodiments of the cell culture systems may be supported from the ground surface using wheels and may define dimensions such that systems described herein may fit through a standard 3 foot wide doorway. In at least one embodiment, the system includes one or more outrigger portions that may be configured to be located in a stowed position when moving the system (e.g., such that the dimensions of the system are under 3 feet to fit through a standard doorway) and located in a deployed support position to assist supporting the system described herein from the ground surface after the system described herein has been moved to its desired location.

In various embodiments, the cell culture systems described herein may be completely self-contained or completely integrated without requiring additional equipment or apparatus, e.g., such as incubators. For example, systems may incorporate, or include, manipulation apparatus, or manipulator, configured to manipulate the position the cell culture vessels (e.g., a powered 2-axis manipulator). By way of further example, the systems described herein may include fully-integrated pumps and valves for dynamic filling/emptying, which may, e.g., be coordinated with the positioning (e.g., rotating, tilting, etc.) of the cell culture vessels will may facilitate rapid processing. Still further, the valves may meter flow therethrough to monitor one or more parameters with respect to stacked cell culture vessels for closed system operation.

Embodiments of the cell culture systems described herein may include monitoring apparatus such as, e.g., one or more sensors, that may be configured to detect the flow rates, fill volumes, temperatures, pressures, etc. with respect to the cell culture vessels. In at least one embodiment, the cell culture systems may be configured to apply and monitor pressure in a cell culture vessel to ensure vessel integrity. Further, in at least one embodiment, the cell culture systems may incorporate, or include, control of temperature and gas concentrations within and/or around the cell culture vessels.

Still further, in various embodiments, the cell culture systems described herein may include one or more human machine interfaces that may be configured to permit a human operator to monitor and adjust automated processes as well as monitor cell culture conditions such as pH, gas concentrations, metabolites, temperature, etc. Further, such human machine interfaces may be located remotely, e.g., such that the physical presence of a human operator local to the system may not be required.

Embodiments of the cell culture systems described herein may, e.g., include, or incorporate, elements to aid closed system processing of appropriately accessorized cell culture vessels, reduce risk of human error and variability through automated processing, provide a non-enzymatic method for cell dissociation, provide a cell dissociation process free from animal derived components, provide less post-dissociation processing of cells that may result in time, labor and equipment savings, and/or include a design configuration that enhances equipment portability and speed of operation.

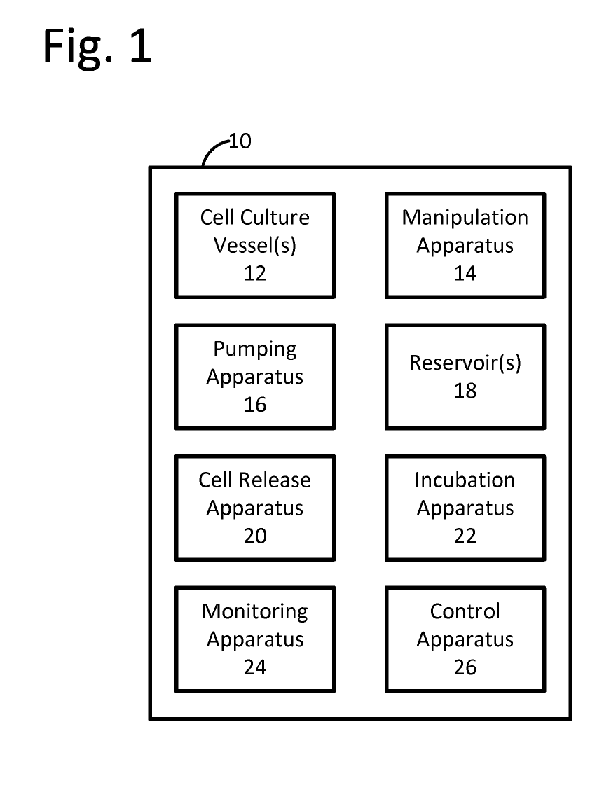

Referring to FIG. 1, a diagram of an embodiment of a cell culture system 10 is depicted. Generally, a cell culture system 10 may include, as shown, one or more cell culture vessels 12, manipulation apparatus 14, pumping apparatus 16, one or more reservoirs 18, cell release apparatus 20, incubation apparatus 22, monitoring apparatus 24, and/or control apparatus 26.

In the depicted embodiment, the cell culture system 10 includes one or more cell culture vessels 12 (e.g., at least one cell culture vessel, more than one cell culture vessel, two or more cell culture vessels, etc.) that may be configured to culture a plurality of anchorage dependent, or adherent, cells. In embodiments, cell culture vessels 12 include a plurality of parallel cell culture surfaces within a plurality of a stacked or multilayer units or compartments (e.g., the plurality cell culture surfaces are parallel to one another). Nonetheless, nearly any cell culture vessel can be adapted for use with systems described herein. For example, any cell culture vessel having a plurality of stacked layers or that can be stacked to form layers can be adapted to be used by the systems described herein. Examples of such cell culture vessels 12 include T-flasks, TRIPLE-FLASK cell culture vessels (Nunc., Intl.), HYPERFLASK cell culture vessels (Corning, Inc.), CELLSTACK culture chambers (Corning, Inc.), CELLCUBE modules (Corning, Inc.), HYPERSTACK cell culture vessels (Corning, Inc.), CELL FACTORY culture apparatuses (Nunc, Intl.), and cell culture articles/vessels as described in WO 2007/015770, entitled “MULTILAYERED CELL CULTURE APPARTUS,” and published Feb. 8, 2007, which is hereby incorporated by reference in its entirety to the extent that it does not conflict with the present disclosure. Of course, cell culture vessels that do not have stacked layers or that are not generally stackable may be used.

In embodiments, cell culture vessels 12 may include a plurality of cell culture surfaces coupled via a manifold. The plurality of culture surfaces may be stacked in a multi-layer configuration. The manifold may include a plurality of fluidly coupled ports that serve to isolate individual or groups of cell culture chambers. Generally, the cell, or growth, culture surfaces are positioned parallel to the ground surface during cell culture processes. To distribute material such as, e.g., cell culture medium, within the cell culture vessels 12, the cell culture vessels 12 may be positioned, or moved, such that the plurality of cell culture surfaces are not positioned parallel to the ground surface such that material may be distributed evenly into all of the chambers/units and across all of the plurality of cell culture surfaces.

A cell culture vessel, or portions thereof, as described herein may be formed from any suitable material. Preferably, materials intended to contact cells or culture media are compatible with the cells and the media. Typically, cell culture units are formed from polymeric material. Examples of suitable polymeric materials include polystyrene, polymethylmethacrylate, polyvinyl chloride, polycarbonate, polysulfone, polystyrene copolymers, fluoropolymers, polyesters, polyamides, polystyrene butadiene copolymers, fully hydrogenated styrenic polymers, polycarbonate PDMS copolymers, and polyolefins such as polyethylene, polypropylene, polymethyl pentene, polypropylene copolymers and cyclic olefin copolymers, and the like.

In some embodiments, the culture vessels (and/or units/compartments therein) contain a gas permeable, liquid impermeable film to allow transfer of gasses between a cell culture chamber and the exterior of the cell culture assembly. Such culture vessels can include spacers or spacer layers positioned adjacent the film, exterior to the chamber, to allow air flow between stacked units. One commercially available example of a cell culture apparatus containing such stacked gas permeable culture units is Corning's HYPERFLASK cell culture apparatus. Such cell culture units may be manufactured in any suitable manner, such as, for example, U.S. Pat. App. Ser. No. 61/130,421, entitled Assembly of Cell Culture Vessels, filed on May 30, 2008 and having Attorney Docket No. 20827, which application is hereby incorporated herein by reference in its entirety to the extent that is does not conflict with the present disclosure. Examples of suitable gas permeable polymeric materials useful for forming a film include polystyrene, polyethylene, polycarbonate, polyolefin, ethylene vinyl acetate, polypropylene, polymethylpentene, polysulfone, polytetrafluoroethylene (PTFE) or compatible fluoropolymer, a silicone rubber or copolymer, poly(styrene-butadiene-styrene) or combinations of these materials. As manufacturing and compatibility for the growth of cells permits, various polymeric materials may be utilized. Preferably the film is of a thickness that allows for efficient transfer of gas across the film. For example, a polystyrene film may be of a thickness of about 0.003 inches (about 75 micrometers) in thickness, though various thicknesses are also permissive of cell growth. As such, the membrane may be of any thickness, preferably between about 25 and 250 micrometers, or between approximately 25 and 125 micrometers. The membrane allows for the free exchange of gases between the chamber of the assembly and the external environment and may take any size or shape. Preferably, the membrane is durable for manufacture, handling, and manipulation of the apparatus.

To distribute material such as, e.g., cell culture media, buffers, proteolytic enzymes, etc. in the cell culture vessels 12, the system 10, as described herein, may include manipulation apparatus 14. Generally, the manipulation apparatus 14 (which may also be referred to as manipulator) can be configured, or be operable, to position the cell culture vessels 12 into one or more different positions to facilitate the culturing processes of anchorage dependent, or adherent, cells within the cell culture vessels 12. Although the particular arrangement of the manipulation apparatus is not limiting, the manipulation apparatus 14 may include holding apparatus that may hold each of the one or more cell culture vessels 12 and movement apparatus that may move the holding apparatus thereby moving the one or more cell culture vessels 12.

For example, the manipulation apparatus 14 may be configured to position the cell culture vessels 12 in one or more filling positions, one or more emptying positions, one or more culturing positions, etc. The one or more filling positions may be defined as positions operable to fill (e.g., effectively fill) the cell culture vessels 12, and likewise, the one or more emptying positions may be defined as positions operable to empty (e.g., effectively empty) the cell culture vessels 12. Further, one or more filling positions may exist since there may be different optimal filling positions for each stage of a filling cycle. For example, the cell culture vessels 12 may be tilted at one or more particular, or selected, angles during the early stage of a filling cycle, and then tilted at one or more particular, or selected, angles during a later stage of the filling cycle different than those in the early stage to effectively fill the cell culture vessels 12. Still further one or more emptying positions may also exist since there may be different optimal emptying positions for each stage of an emptying cycle. For example, the cell culture vessels 12 may be tilted at one or more particular, or selected, angles during the early part of an emptying cycle, and then tilted at one or more particular, or selected, angles during the latter part of the emptying cycle different than those in the early stage to effectively empty the cell culture vessels 12. The one or more culturing positions may generally include positions in which the cell culture, or growth, surfaces are parallel to a ground surface (e.g., to facilitate effective cell growth). Further, although a few different positions are described herein, fill positions, emptying positions, and incubation positions/conditions may be specific to the particular cell culture vessel used, and as such, the systems described herein may operate differently to accommodate the particular cell culture vessels being used. In other words, the fill positions, emptying positions, and incubation positions/conditions described herein are not the only positions that the systems described herein are capable of, and further, the systems described herein may be configured to accommodate the positions used for any particular cell culture vessel.

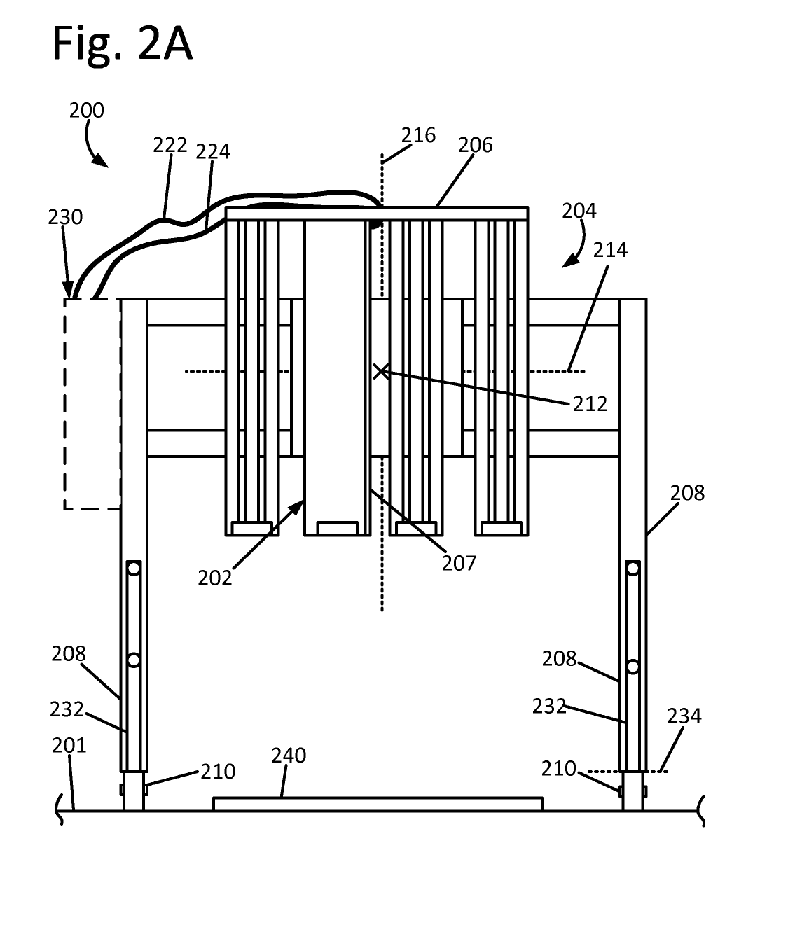

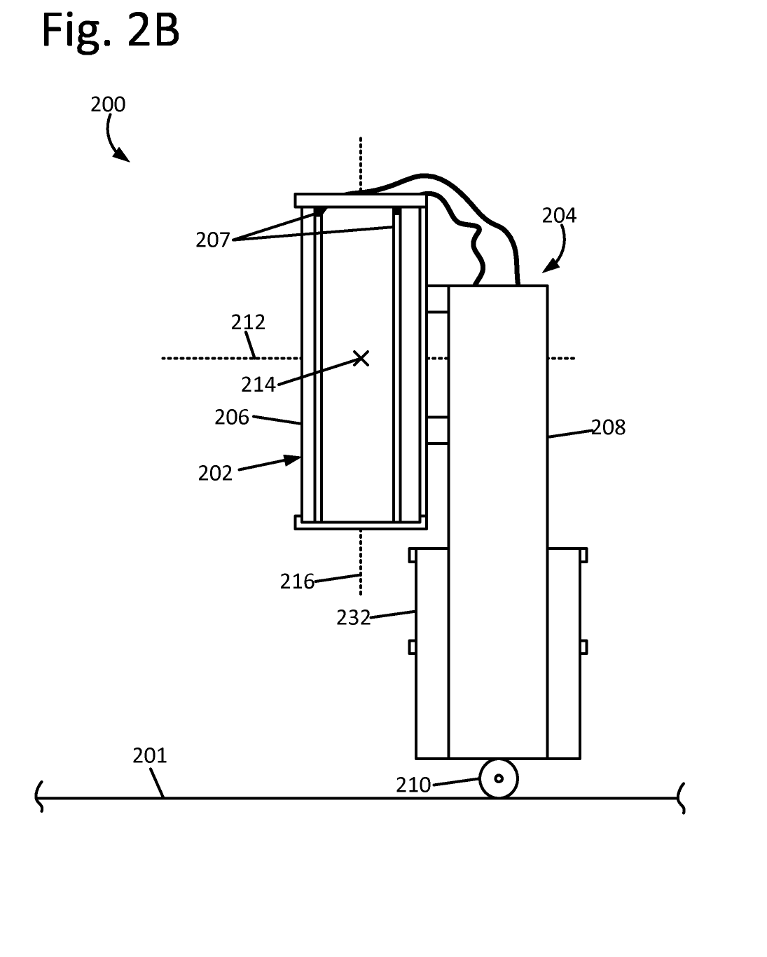

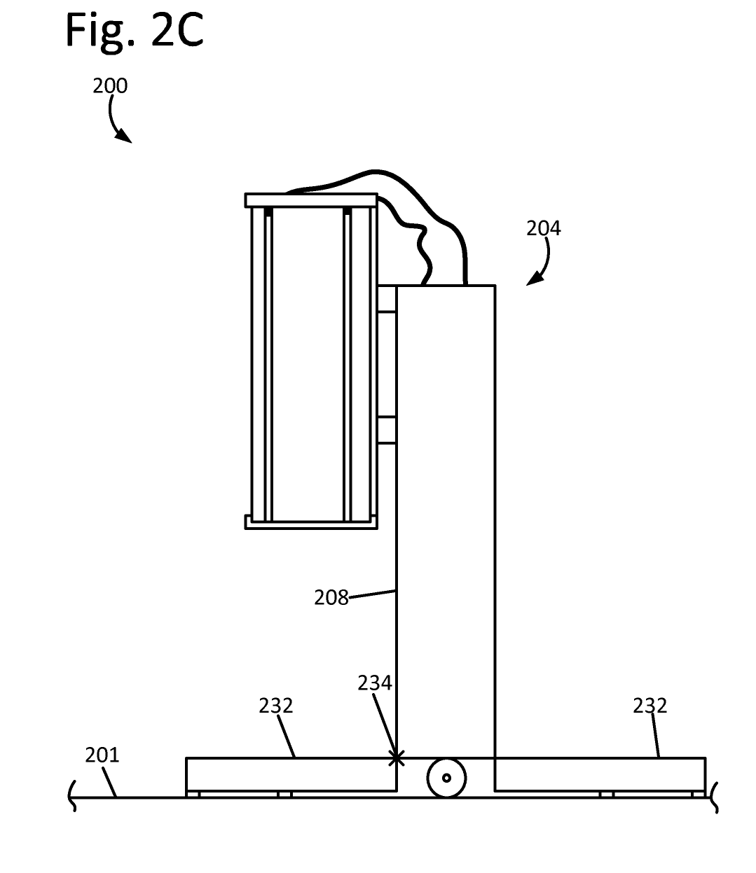

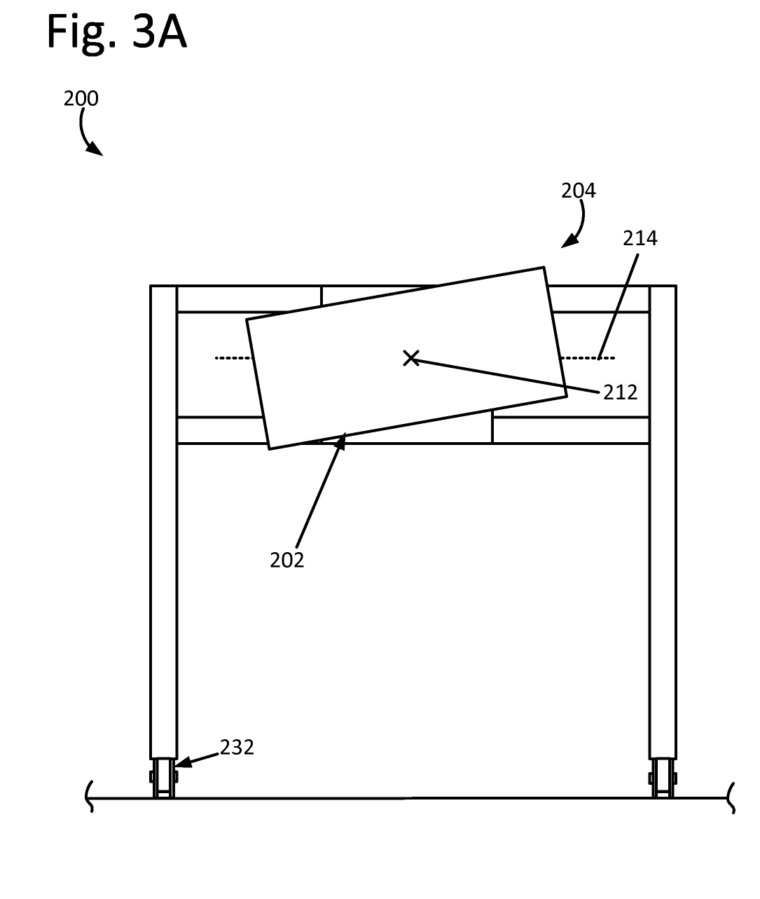

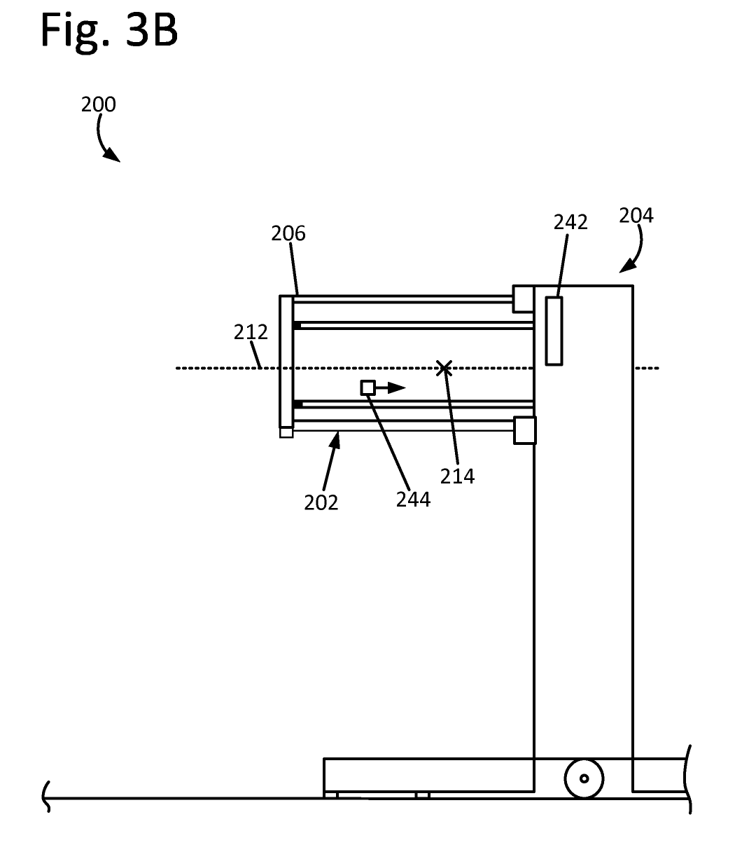

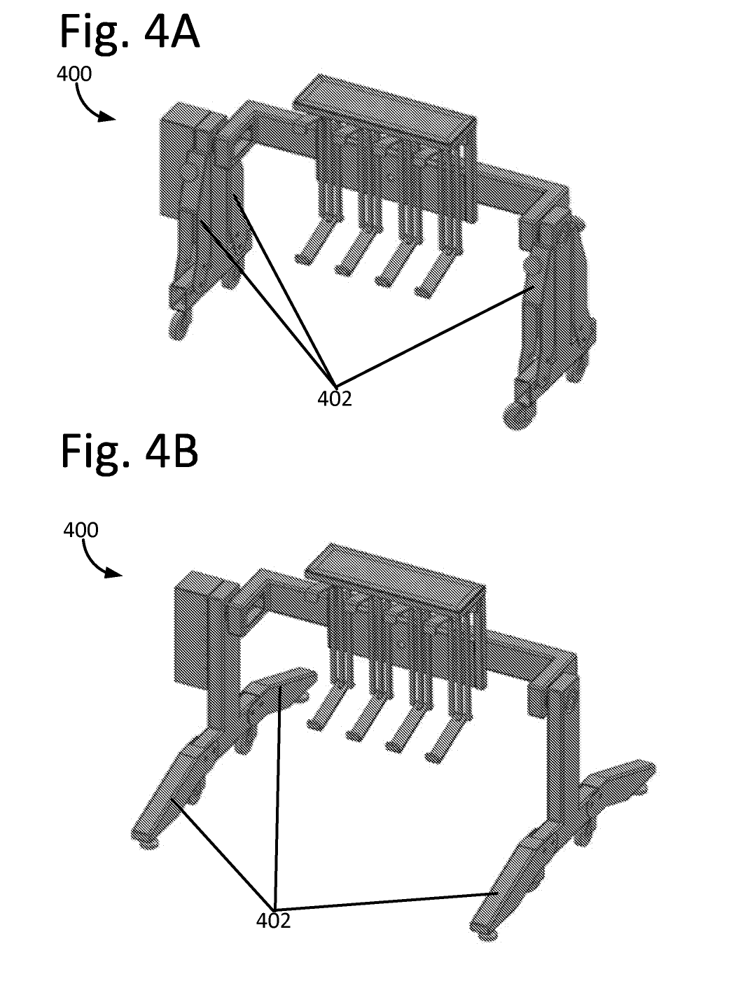

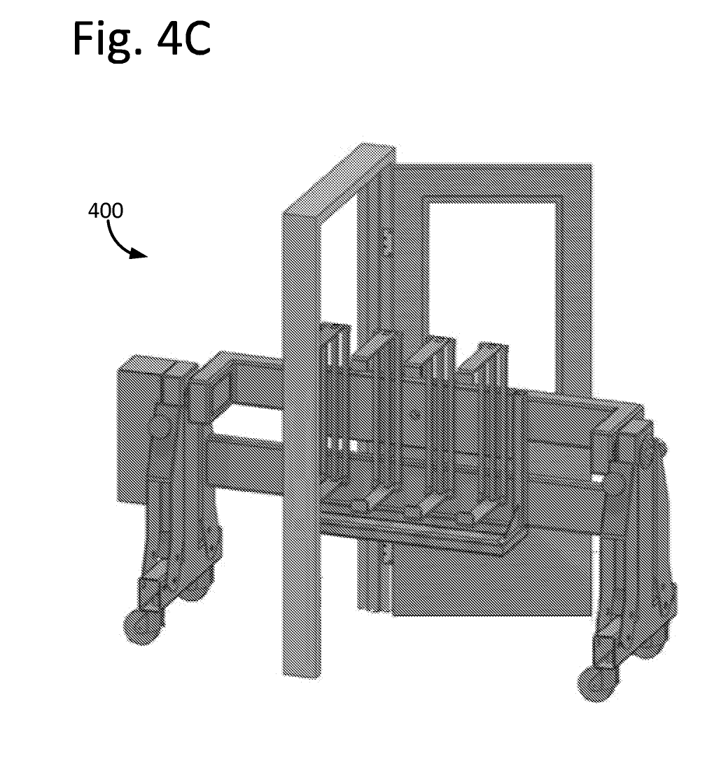

In embodiments, the manipulation apparatus 14 may be configured to move the cell culture vessels 12 about a first axis and a second axis, each of the first axis and the second axis being perpendicular to one another and parallel to the ground surface (upon which manipulation apparatus 14 is located). In embodiments, the manipulation apparatus 14 may be configured to move the cell culture vessels vertically along a vertical axis to, for example, assist the loading and unloading of the cell culture vessels 12 into or onto other various apparatus of the system 10 for use thereof. One embodiment of manipulation apparatus 14 will be described further herein with reference to FIGS. 2-3, and another embodiment of manipulation apparatus 14 will be described further herein with reference to FIGS. 4A-4C.

A cell culture system 10 may include pumping apparatus 16 that may be configured to pump material such as, e.g., cell culture medium, into and out of the cell culture vessels 12 through at least one port located on each of the cell culture vessels 12. For example, each of the cell culture vessels 12 may include a manifold fluidly coupling each of the cell culture compartments or units of the cell culture vessel 12 to at least one port such that materials may be pumped into and out of cell culture vessels 12 using the at least one port.

The pumping apparatus 16 may be fluidly coupled to each of the cell culture vessels 12. In at least one embodiment, the pumping apparatus 16 may include at least one pump for each of the cell culture vessels 12 to, e.g., maintain a closed system, prevent cross contamination when using one pump for multiple cell culture vessels, etc. In other words, the pumping apparatus 16 may include a plurality of pumps. Further, the pumping apparatus 16 may include a plurality of valves which may be used to selectively connect, or fluidly couple, one or more reservoirs 18 to the pumping apparatus 16 such that materials located within the reservoirs 18 may be pumped into the cell culture vessels 12 and/or materials located within the cell culture vessels 12 may be pumped into the reservoirs 18. Each reservoir 18 may be defined as a fluid tight container, or vessel, configured to hold material.

As used herein, “material,” e.g., that is pumped into and out of the cell culture vessel 12 and the reservoirs 18, may be defined as any flowable material (e.g., liquid) that may be used in cell culture processing. For example, material may include cell culture medium (e.g., containing cells to be cultured), spent medium, proteolytic enzymes, quench solutions, chelating solutions, buffers, transfection agents, etc.

The pumping apparatus 16 and reservoirs 18 may be coupled to the manipulation apparatus 14 and/or any other portion of the cell culture system 10 such that the pumping apparatus 16 and the reservoirs 18 may be integral, or self-contained, within the cell culture system 10.

A cell culture system 10 may further include cell release apparatus 20. Generally, cell release apparatus 20 may be operable to release cells adhered, attached, or anchored to the cell culture, or growth, surfaces of the cell culture vessels 12, e.g., after the cells have been cultured. In at least one embodiment, the cell release apparatus 20 may include shaking apparatus configured to shake the cell culture vessels 12 at a frequency greater than or equal to about 0.1 kHz, about 0.5 kHz, about 1 kHz, etc. and/or less than or equal to about 5 kHz, about 10 kHz, about 15 kHz, about 20 kHz, etc. to release at least a portion of a plurality of cells adhered to the cell culture surfaces of cell culture vessels 12. In at least one embodiment, the cell release apparatus 20 may include shaking apparatus configured to shake the cell culture vessels 12 at an amplitude of about 12 millimeters (mm) to about 26 mm. Further, the shaking path can be oriented at a wide range of angles relative to the cell culture surfaces of the cell culture vessels 12. For example, the shaking apparatus may be configured to move the cell culture vessels 12 in a circular path, vertically, parallel to the cell culture surfaces, and in linear reciprocation. The shaking apparatus may be as described in U.S. Prov. Pat. App. Ser. No. 61/527,164 (Corning No. SP 11-201) entitled “METHODS OF RELEASING CELLS ADHERED TO A CELL CULTURE SURFACE” and filed Aug. 25, 2011, which is incorporated herein by reference in its entirety to the extent it does not conflict with the disclosure presented herein. The shaking apparatus may be integral or separate from the manipulation apparatus 14. For example the shaking apparatus may be coupled to the manipulation apparatus 14 and configured to shake at least a portion of the manipulation apparatus 14 such that the cell culture vessels 12 held by the manipulation apparatus 14 may shake. Further, for example, the shaking apparatus may be located apart, away, or separately from the manipulation apparatus 14. In this example, the manipulation apparatus 14 or the shaking apparatus may move with respect to the other to locate the shaking apparatus and cell culture vessels 12 in contact with one another such that the shaking apparatus can shake the cell culture vessels 12 to release at least a portion of a plurality of cells adhered to the cell culture surfaces of the cell culture vessels 12. In embodiments, the shaking apparatus may be configured to be in contact with, or in close proximity to, at least a portion of the cell culture vessels 12 and slide across, or relative to, the cell culture vessel 12 so as to deliver shaking energy to portions of the cell culture vessel 12 as the transducer slides relative to the vessel 12.

In embodiments, the shaking apparatus may include a platform upon which the cell culture vessels 12 may be placed by the manipulation apparatus 14. After the cell culture vessels 12 have been placed on the platform, the platform may shake thereby shaking the cell culture vessels 12 to release at least a portion of a plurality of cells adhered to the cell culture surfaces of the cell culture vessels 12. After the cell culture vessels 12 have been shaken by the shaking apparatus, the manipulation apparatus 14 may pick up and move the cell culture vessels 12 from the platform.

In embodiments, cell release apparatus 20 may include ultrasonic transducer apparatus configured to provide ultrasonic energy to the cell culture vessels 12 at a frequency greater than or equal to about 1 kHz, about 10 kHz, about 15 kHz, etc. and less than or equal to about 20 kHz, about 30 kHz, about 40 kHz, etc. Further, the ultrasonic transducer apparatus may be configured to provide ultrasonic energy to the cell culture vessels 12 for about 5 seconds to about 30 seconds for each cell culture vessel one or more times. For example, the ultrasonic transducer apparatus may be as described in U.S. Pat. App. Pub. No. 2009/0298153 entitled “METHOD FOR ULTRASONIC CELL REMOVAL,” published on Dec. 3, 2009, and filed on May 19, 2009, which is also incorporated herein by reference in its entirety to the extent that it does not conflict with the disclosure presented herein. Further, ultrasonic transducer apparatus may be configured to be movable with respect to the cell culture vessels 12 and/or the manipulation apparatus 14 so as to be able to deliver ultrasonic energy to at least one of the one or more chambers, units, or compartments of the cell culture vessels 12. For example, the ultrasonic transducer apparatus may be configured to be in contact with, or in close proximity to, at least a portion of the cell culture vessels 12 and slide across, or relative to, the cell culture vessel 12 so as to deliver ultrasonic energy to portions of the cell culture vessel 12 as the transducer slides relative to the vessel 12. Further, for example, the ultrasonic transducer apparatus may be configured to be directional such that it may direct, or sweep, the ultrasonic energy across the cell culture vessel 12, using, e.g., a horn.

A cell culture system 10 described herein may further include incubation apparatus 22. The incubation apparatus 22 may be generally described as any apparatus capable of incubating the cell culture vessels 12 to facilitate incubation of the cells within the cell culture vessels 12. For example, the incubation apparatus 22 may apply heat to the cell culture vessels 12 in the range of 30 degrees Celsius to about 40 degrees Celsius. In at least one embodiment, the incubation apparatus 22 may completely surround the manipulation apparatus 14. In at least another embodiment, the incubation apparatus 22 may be apart from the manipulation apparatus 14 such that the manipulation apparatus 14 may position, or move, the cell culture vessels 12 into the incubation apparatus 22 for incubation and/or out of the incubation apparatus 22 after incubation.

A cell culture system 10 described herein may include monitoring apparatus 24. Generally, monitoring apparatus 24 may be configured to monitor any one or more parameters associated with the cell culture system 10. For example, the monitoring apparatus 24 may be configured to monitor one or more of the cell culture vessels 12, the manipulation apparatus 14, the pumping apparatus 16, the reservoirs 18, the cell release apparatus 20, the incubation apparatus 22, etc. Further, the monitoring apparatus 24 may include position sensors, temperature sensors, pressure sensors, light sensors, fill position sensors, oxygen sensors, carbon dioxide sensors, pH sensors, gas concentration sensors, fluorescent-imaging based sensors, optical sensors, glucose sensors, lactate sensors, ammonium sensors, load cells (e.g., for weighing cell culture vessels), electrical impedance sensors, ultrasonic impedance sensors, vision systems, and/or any other sensor that may be used in the cell culture system 10.

The monitoring apparatus 24 may be used by the control apparatus 26 (described further herein) of a cell culture system 10 to monitor the cell culture system 10 to provide feedback for adjusting one or more parameters with respect the cell culture system 10.

In embodiments, position sensors of the monitoring apparatus 24 may be configured to monitor the position of the cell culture vessels 12 such as, e.g., the rotation of the cell culture vessels 12 about a first axis parallel a ground surface, the rotation of the cell culture vessels 12 about a second axis parallel to ground surface, the distance of the cell culture vessels 12 above the ground surface, etc. Such position data may be used, e.g., by the control apparatus 26, to confirm movements made to the cell culture vessels 12 using the manipulation apparatus 14. In at least another embodiment, temperature sensors of the monitoring apparatus 24 may be configured to monitor the temperature inside or outside of the cell culture vessels 12 and/or the temperature inside the incubator apparatus 22. Such temperature data may be used for monitoring purposes and/or adjusting of the incubator apparatus.

In embodiments, pressure sensors of monitoring apparatus 24 may be configured to measure the pressure within each cell culture vessel 12, each reservoir 18, and/or the incubation apparatus 22. In at least one embodiment, fill level, or position, sensors of the monitoring apparatus 24 may be configured to monitor the amount of material (e.g., the fill level) within the cell culture vessels 12 or the reservoirs 18. Such fill level data may be used to determine if the cell culture vessels 12 are full. In at least one embodiment, oxygen sensors of the monitoring apparatus 24 may be configured to monitor the oxygen concentration within the cell culture vessels 12, the reservoirs 18, or the incubation apparatus 22, and carbon dioxide sensors of the monitoring apparatus 24 may be configured to monitor the carbon dioxide concentration within the cell culture vessels 12, the reservoirs 18, or the incubation apparatus 22. In embodiments, the control apparatus 26 may be configured to modify the rate at which material is pumped into and out of each culture vessel 12 using the pumping apparatus 16 based on the one or more monitored parameters of the culture vessels 12.

In embodiments, the optical sensors of the monitor apparatus 24 may be configured to image the material within the cell culture vessels 12 (e.g., image the cell culture medium, etc.) and the control apparatus 26 may be configured to provide the images to a user. Further, the user may be remote from the system 10, e.g., such that the user can view images of the cell culture without being located local, or near, the system 10. In other words, the cell culturing system 10 can provide remote visualization of the cell culture (e.g., which may provide rapid assessment of cell confluence). Further, after the cells are released from the cell culture surfaces, the cells could be also checked using such remote visualization. In effect, optical sensors of the monitoring apparatus 24 may provide a remote microscope to view the cell cultures.

The control apparatus 26 of the cell culture system 10 may include one or more computing devices capable of processing data. The control apparatus 26 may include, e.g., microprocessors, programmable logic arrays, data storage (e.g., volatile or non-volatile memory and/or storage elements), input devices, output devices, etc. The control apparatus 26 may be programmed to implement the methods or portions of the methods described herein and may be operably coupled to each element of the cell culture system 10 to, e.g., monitor or adjust one or more parameters with respect to each element of the cell culture system 10. For example, the control apparatus 26 may be operably coupled to the cell culture vessels 12, the manipulation apparatus 14, the pumping apparatus 16, the reservoirs 18, the cell release apparatus 20, the incubation apparatus 22, or the monitoring apparatus 24.

As described herein, “operably coupled” may be defined as connected (e.g., wired or wirelessly) such that information (e.g., image data, commands, etc.) may be transmitted between each object.

The methods described herein may be implemented by program code or logic. Program code or logic described herein may be applied to input data to perform functionality described herein and generate desired output information. The output information may be applied as input to one or more other devices and/or processes as described herein or as would be applied in a known fashion.

The program code or logic used to implement the present invention may be provided using any programmable language, e.g., a high level procedural or object orientated programming language that is suitable for communicating with controller apparatus. Any such program code or logic may, for example, be stored on any suitable device, e.g., a storage media, readable by a general or special purpose program, computer or a processor apparatus for configuring and operating the computer when the suitable device is read for performing the procedures described herein. In other words, at least in one embodiment, the control apparatus 26 may be implemented using a non-transitory computer readable storage medium, configured with a computer program, where the storage medium so configured causes the control apparatus 26 to operate in a specific and predefined manner to perform functions described herein.

At least in one embodiment, the control apparatus 26 may be, for example, any fixed or mobile computer system (e.g., a personal computer or minicomputer). The exact configuration of the control apparatus 26 is not limiting and essentially any device capable of providing suitable computing capabilities may be used according to the present invention.

In view of the above, it will be readily apparent that the functionality as described in one or more embodiments according to the present invention may be implemented in any manner as would be known to one skilled in the art. As such, the computer language, the controller apparatus, or any other software/hardware which is to be used to implement the present invention shall not be limiting on the scope of the processes or programs (e.g., the functionality provided by such processes or programs) described herein.

Generally, the control apparatus 26 may be configured to initiate or control one or more element of the cell culture system 10 to facilitate the automatic, or automated, culturing of cells within, e.g., a closed system to minimize contamination, etc. For example, the control apparatus 26 may be configured to control the manipulation apparatus 14 to move the cell culture vessels 12 into various positions such as, e.g., filling positions, emptying positions, culturing positions, etc. An embodiment of a manipulation apparatus positioning a cell culture vessel in a horizontal culturing position will be described further herein with reference to FIGS. 2A-2C and in a filling position will be described further herein with reference to FIGS. 3A-3B.

The control apparatus 26 may be configured to control the pumping apparatus 16 to pump material (e.g., cell culture medium) from the reservoirs 18 to the cell culture vessels 12 or to pump material (e.g., spent medium, harvested cells, etc.) from the cell culture vessels 12 to the reservoirs 18. In at least one embodiment, the control apparatus 26 may be configured to control the manipulation apparatus 14 and the pumping apparatus 16 at the same time so as to move the cell culture vessels 12 into a filling position and at the same time use the pumping apparatus 16 to move material from the reservoirs 18 to the cell culture vessels 12 thereby facilitating an automatic fill process. In embodiments, the control apparatus 26 may be configured to control the manipulation apparatus 14 and pumping apparatus 16 at the same time so as to move the cell culture vessels 12 into an emptying position at the same time as controlling the pumping apparatus 16 to move material from the cell culture vessels 12 to the reservoirs 18 thereby facilitating an automatic emptying process. For example, while the cell culture vessels 12 may be moved into an emptying position, the pumping apparatus 16 may simultaneously begin pumping to move material from the cell culture vessels 12 to the reservoirs 18 (or from the reservoirs 18 to the cell culture vessels 12). In other words, material may be pumped out of the cell culture vessels 12 while the cell culture vessels 12 are moving into an emptying position or material may be pumped into the cell culture vessels 12 while the cell culture vessels 12 are moving into a filling position. Further, the movement of the cell culture vessels 12 may be coordinated with the pumping such the position of the cell culture vessels 12 at any given moment is optimal for the pumping conditions or parameters.

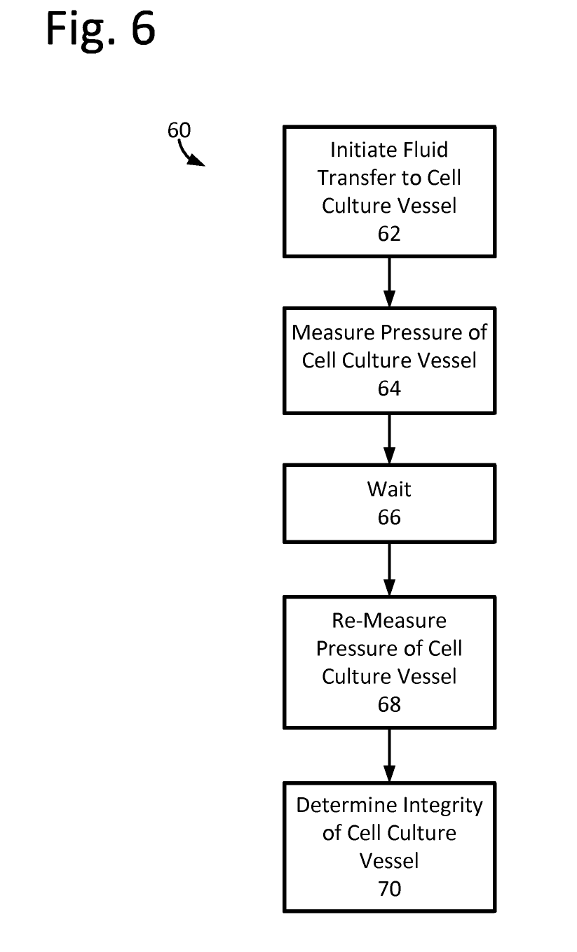

In at least one embodiment, the control apparatus 26 may be configured to apply pressure to a cell culture vessel 12 using the pumping apparatus 16 and to monitor the pressure of the cell culture vessel 12 to verify the integrity of the cell culture vessel 12 (e.g., confirm that the cell culture vessel 12 is airtight or sealed, confirm that the cell culture vessel 12 is not leaking, etc.). An embodiment of a method of testing the integrity of a cell culture vessel 12 is described further herein with reference to FIG. 6.

An embodiment of a cell culture system 200 is depicted in FIGS. 2A-2C & 3A-3B. The depicted cell culture system 200 includes a cell culture vessel 202 and manipulation apparatus 204. The manipulation apparatus 204 includes holding apparatus 206, which as depicted includes four sets of jaws, each set of jaws configured to hold a cell culture vessel 202 (although only one cell culture vessel 202 is depicted). Further, although not shown, the holding apparatus 206 may be configured to “open” to receive the cell culture vessel 202 and to close to hold the cell culture vessel 202 (e.g., the jaws may open away from each other and close towards each other to pinch the cell culture vessel 202 therebetween). As shown, the manipulation apparatus 204 is only holding a single cell culture vessel 202 but may hold up to four. In other embodiments, the manipulation apparatus 204 may hold less than four cell culture vessels or more than four cell culture vessels. The manipulation apparatus 204 further includes legs 208 configured to support the cell culture vessel 202 off of the ground surface 201. Each of the legs 208 may include wheels 210 located proximate the bottom of the leg 208 to provide ease of transport of the system 200 by rolling it along a ground surface 201.

The manipulation apparatus 204 may be configured to rotate, pivot, or move, the cell culture vessel 202 about at least two axes 212, 214 (also referred to as a first axis 212 and a second axis 214). Each of the two axes 212, 214 are parallel to the ground surface 201, and each of the two axes 212, 214 are perpendicular to each other. In at least one embodiment, the manipulation apparatus 204 may be configured to rotate the cell culture vessel 202 about axis 212 from about negative 10 degrees to about positive 100 degrees from horizontal (or parallel the ground surface 201). In at least one embodiment, the manipulation apparatus 204 may be configured to rotate the cell culture vessel 202 about axis 214 from about negative 10 degrees to about positive 90 degrees from horizontal (or parallel the ground surface 201). Further, manipulation apparatus may be configured to move the cell culture vessel 202 along a vertical axis 216 (e.g., to load and unload the cell culture vessel 202 from a surface such as the ground surface 201, a scale, a platform for applying shaking or vibration for cell release, etc.).

The cell culture vessel 202 is arranged in a horizontal cell culturing position as shown in FIGS. 2A-2C. In the horizontal cell culturing position, the cell culture, or growth, surfaces of the cell culture vessel 202 are arranged to be parallel to the ground surface 201 to, e.g., facilitate cell growth on the cell culture, or growth, surfaces. As described herein, the manipulation apparatus 204 is configured to move the cell culture vessel 202 into other positions such as one or more filling positions or one or more emptying positions. For example, the manipulation apparatus 204 has moved the cell culture vessel 202 into a filling position in FIGS. 3A-3B. As shown in FIGS. 3A-3B, the cell culture vessel 202 has been rotated negative 90° about axis 214 (counterclockwise as shown in FIG. 3B) and about 10° about axis 212 (counterclockwise as shown in FIG. 3A) for the filling position. The filling position shown in FIGS. 3A-3B positions the cell culture, or growth, surfaces of the cell culture vessel 202 perpendicular to the ground surface 201 and positions the end of the cell culture vessel 202 opposite from a manifold 207 of the cell culture vessel 202 to a position lower than the manifold 207 so as to, e.g., facilitate equal distribution of material introduced into the cell culture vessel 202 via the manifold 207.

Conversely, although not shown, the manipulation apparatus 204 may be configured in an emptying position by rotating the cell culture vessel 202 negative 90° about axis 214 (counterclockwise as shown in FIG. 3B) and about negative 10° about axis 212 (clockwise as shown in FIG. 3A). Such emptying position may position the cell culture, or growth, surfaces of the cell culture vessel 202 perpendicular to the ground surface 201 and position the manifold 207 lower than the opposite end of cell culture vessel 202 so as to, e.g., facilitate emptying of material out of the cell culture vessel 202 via the manifold 207.

The dashed portion 230 of the depicted cell culture system 200 is representative of any other element, or portion, of the system 200 not graphically depicted in FIGS. 2A-2C & 3A-3B but described herein with reference to any cell culture system (e.g., with reference to cell culture system 10). For example, the portion 230 may include pumping apparatus, reservoirs, monitoring apparatus, cell release apparatus, control apparatus, etc. As shown, hoses 222 and 224 fluidly couple the manifold 207 of the cell culture vessel 202 (and, in turn, the cell culture vessel 202 itself) to the pumping apparatus within the portion 230. In at least one embodiment, hose 222 is used for pumping material into and out of the cell culture vessel 202 and hose 224 is used for venting purposes.

The cell culture system 200 may further include outrigger portions 232, or outriggers, that are positionable in a stowed configuration, or position, as shown in FIGS. 2A-2B and in a deployed support configuration, or position, as shown in FIG. 2C. The outrigger portions 232 may provide additional support and stability to the cell culture system 200 when in the deployed configuration. Further, the outrigger portions 232, when in the stowed configuration, may provide dimensions for the cell culture system 200 such that the system 200 may pass through a standard 3 feet wide door. In at least one embodiment, the outrigger portions 232 may be pivotable about an axis 234 from the deployed position to the stowed position or from the stowed position to the deployed position.

FIGS. 4A-C show a schematic conceptual design of an automated cell culture system 400 including outriggers 402, or outrigger portions, that may be moved between a stowed configuration and a deployed configuration. For example, the outriggers 402 are configured in a stowed configuration in FIG. 4A to permit the system 400 to pass through a standard 3 foot wide doorway (e.g., as shown in FIG. 4C). Further, for example, the outriggers 402 are configured in deployed, or active outward, configuration in FIG. 4B to add stability to the system 400 once it is located where it will function. Further, the system 400 shown in FIGS. 4A-C may incorporate, or include, any one or more features or apparatus of the cell culture systems described herein.

Embodiments of cell release apparatus are depicted in FIGS. 2A & 3B. For example, shaking apparatus 240 that includes a platform is depicted in FIG. 2A. The cell culture vessel 202 may be lowered onto to the platform of the shaking apparatus or ultrasonic transducer apparatus 240 and the shaking apparatus or ultrasonic transducer apparatus 240 may shake or deliver ultrasonic energy to the platform to release at least a portion of a plurality of cells adhered to the cell culture surfaces of the cell culture vessel 202.

Further, for example, shaking apparatus or ultrasonic transducer apparatus 242 that is coupled to the manipulation apparatus 204 is depicted in FIG. 3B. In one embodiment, the shaking apparatus or ultrasonic transducer apparatus 242 may be configured to shake or deliver ultrasonic energy to at least a portion of the manipulation apparatus 204 to release at least a portion of a plurality of cells adhered to the cell culture surfaces of the cell culture vessel 202. In embodiments, the apparatus 242 or the manipulation apparatus 204 may move with respect to each other such that the cell culture vessel 202 becomes in contact with the apparatus 242 such that the apparatus 242 may shake or deliver ultrasonic energy to the cell culture vessel 202 to release at least a portion of a plurality of cells adhered to the cell culture surfaces of the cell culture vessel 202.

Further, for example, shaking apparatus or ultrasonic transducer apparatus 244 is depicted in FIG. 3B. As shown, the shaking apparatus or ultrasonic transducer apparatus 244 may be movably coupled to the manipulation apparatus 204 and configured to move about the cell culture vessel 202 (as represented by the arrow) to shake or deliver ultrasonic energy to the cell culture vessel 202 to release at least a portion of a plurality of cells adhered to the cell culture surfaces of the cell culture vessel 202.

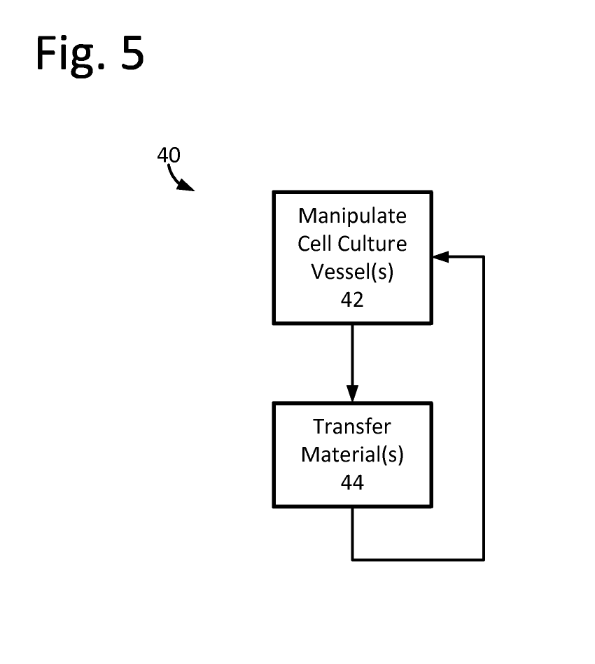

An embodiment of a material transfer method 40 for use with a cell culture systems described herein is depicted in FIG. 5. The method 40 may include manipulating one or more cell culture vessels 42 and transferring material 44 into or out of the one or more cell culture vessels 42 either during or after the manipulation 42. For example, the cell culture vessels may be manipulated into a filling position 42, and after being manipulated to the filling position 42, the method 40 may initiate the transfer of materials 44 such as, e.g., cell culture medium, etc., from one or more reservoirs to the cell culture vessels. Further, for example, the cell culture vessels may be manipulated into an emptying position, and after being manipulated into the emptying position 42, the method 40 may initiate the transfer of materials 44 such as, e.g., spend medium, harvested cells, etc., from the cell culture vessels to one or more reservoirs.

Filling and emptying methods for use with cell culture systems described herein may include multiple different positions to facilitate the filling and emptying methods. As such, the method 40 to continually, periodically, or on an as-needed basis, manipulate the cell culture vessels 42 (e.g., into one or more positions) while transferring materials 44 to/from the cell culture vessels 42 (as shown by the arrow looping back from process 44 to process 42). In at least one embodiment, the cell culture vessels may be manipulated 42 and materials may be transferred 44 at the same time. In embodiments, one or more sensor may detect fill level of the culture vessel to provide feedback to a control unit for purposes of manipulating the vessel to the appropriate position during the filling or emptying process. Any suitable sensor may be used to detect fill level of the vessel during the filling or emptying process. In embodiments, a load sensor or other mass sensor may be used to measure the mass of each of the at least one cell culture vessel, e.g., for purposes of detecting fill level (e.g., the data from the load sensor or other mass sensor may be used in coordinating the movement of the at least one cell culture vessel using the manipulation apparatus with the pumping of material into and out of the at least one culture vessel using the pumping apparatus). In embodiments, one or more optical sensor, infrared sensor, or the like may be suitably positioned along the culture vessel to detect fill level.

An embodiment of a cell culture vessel integrity check method 60 for use with the cell culture systems described herein is depicted in FIG. 6. The depicted method 60 may be used after loading a cell culture vessel into the manipulation apparatus and connecting pumping apparatus to the cell culture vessel to check the integrity of the cell culture vessel. In other words, the method 60 may be used to determine if the cell culture vessel has been compromised or is leaking. Further, the method 60 may be used to determine if the cell culture vessel has been properly coupled to the pumping apparatus of a cell culture system.

The depicted method 60 may first initiate a fluid transfer (e.g., air, etc.) to the cell culture vessel 62 for about 1 minute to about 2 minutes per cell culture vessel using, e.g., pumping apparatus of a cell culture system, and then measure the pressure of the cell culture vessel 64 using, e.g., a pressure sensor of the monitoring apparatus of a cell culture system. The method 60 may then wait 66 for a selected period of time, e.g., for stabilization, (e.g., a selected period of time greater than or equal to about 1 minute, about 2 minutes, about 2.5 minutes, etc. and/or less than or equal to about 5 minutes, 4 minutes, about 3 minutes, etc.), then the pressure of the cell culture vessel may then be measured 68 for about 15 seconds, and then the integrity of the cell culture vessel (or, as described herein, the proper coupling of the cell culture vessel to the pumping apparatus) may be determined 67. To determine the integrity of the cell culture vessel 70, the pressure values measured, or taken, during process 64 and process 68 may be evaluated. For example, the integrity of the cell culture vessel 70 determined by any monitored pressure decay. Further, for example, if the two pressure values are substantially different, the cell culture vessel may have been compromised, may be leaking, and/or has not been coupled properly to the pumping apparatus. In at least one embodiment, the difference between the two measured pressure values may be compared to a threshold value. In other words, to test the integrity of the cell culture vessel, the method 60 may apply pressure to the cell culture vessel and may monitor the pressure to determine whether the cell culture vessel has been compromised.

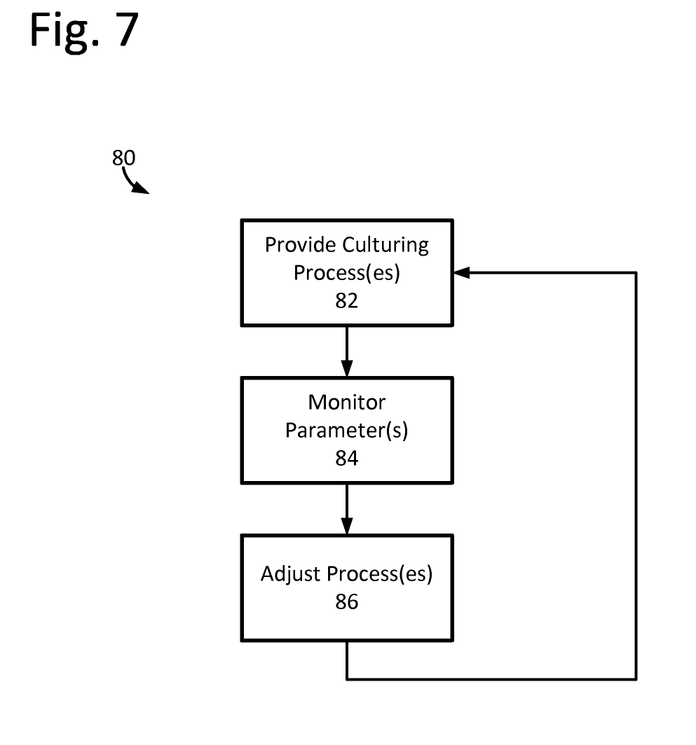

An embodiment of a cell culturing and monitoring method 80 for use with the cell culture systems described herein is depicted in FIG. 7. The method 80 may include providing one or more culturing processes 82 (e.g., filling, emptying, harvesting, incubating, releasing cells from cell culture surfaces, etc.), and during the processes 82, the method 80 may continually monitor one or more parameters 84 with respect to the culturing processes 82 and adjust the culturing processes 86 based on the one or more monitored parameters 82. In other words, the method 80 provides a closed loop feedback cycle for automatically adjusting one or more cell culturing processes within the cell culture systems described herein.

For example, during an incubation process 82, the method 80 may monitor the temperature in the incubation apparatus 84 and adjust the temperature in the incubation apparatus 86 (e.g., higher or lower) based on the monitored temperature. Further, for example, during a filling process 82, the method 80 may monitor the fill volume (or level) of a cell culture vessel (or reservoir) 84 and adjust the fill volume using pumping apparatus 86 (e.g., more or less material) based on the monitored fill volume.

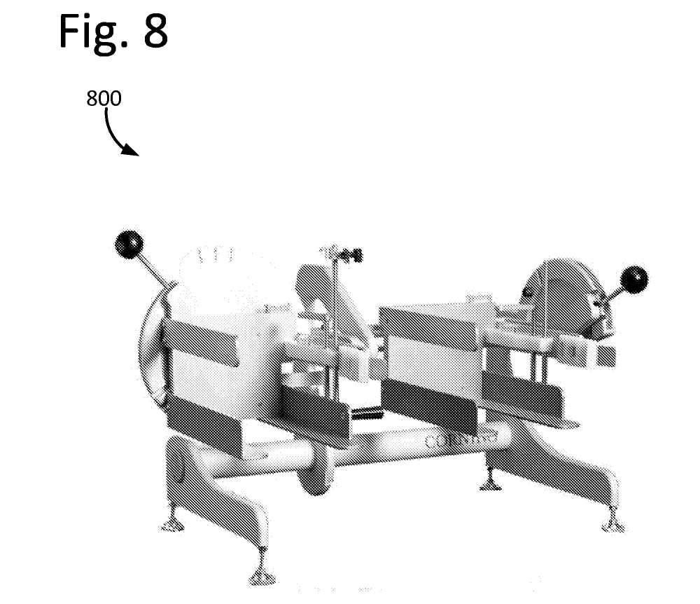

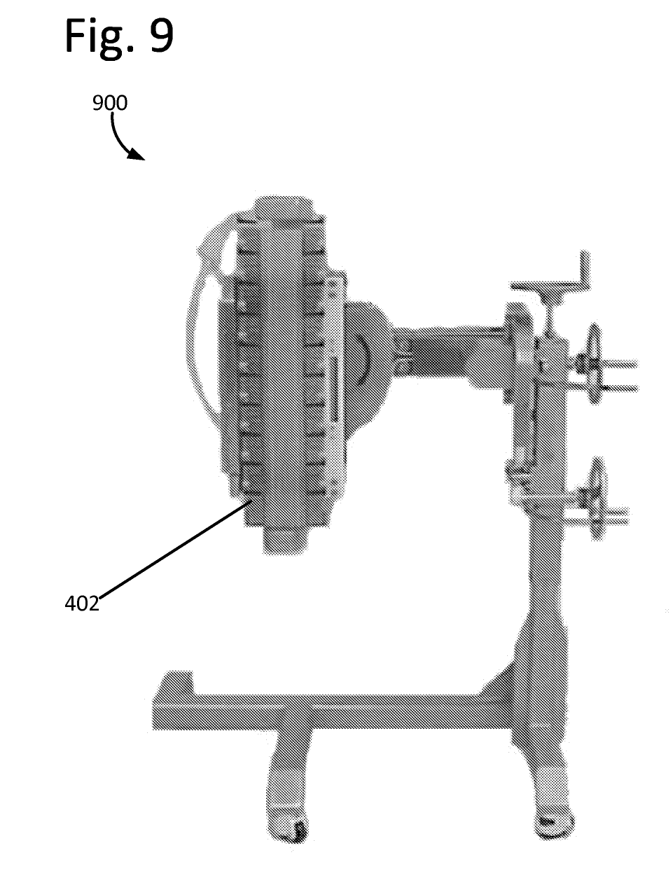

Further, one aspect of the present disclosure is to incorporate cell release apparatus such as, for example, shaking apparatus described in U.S. Prov. Pat. App. Ser. No. 61/527,164 (Corning No. SP 11-201) entitled “METHODS OF RELEASING CELLS ADHERED TO A CELL CULTURE SURFACE” and filed Aug. 25, 2011, which is incorporated herein by reference in its entirety to the extent that it does not conflict with the disclosure presented herein, and ultrasonic transducer apparatus described in U.S. Pat. App. Pub. No. 2009/0298153 entitled “METHOD FOR ULTRASONIC CELL REMOVAL,” published on Dec. 3, 2009, and filed on May 19, 2009, which is also incorporated herein by reference in its entirety to the extent that it does not conflict with the disclosure presented herein, into a manual stacked cell culture manipulator. For example, a manual cell culture vessel manipulator 800 configured for desktop use is shown in FIG. 8, which may be modified to incorporate cell release apparatus as well as any other various apparatus and equipment described herein. By way of further example, another manual cell culture vessel manipulator 900 is shown in FIG. 9, which may also be modified to incorporate cell release apparatus as well as any other various apparatus and equipment described herein. As shown, a Corning HYPERSTACK 902 is being held upright by the manipulator 900.

Further, cell release apparatus may also be incorporated into automated cell culture vessel handling systems such as, e.g., the TAP Biosystems SELECT or COMPACT systems. For example, a vessel nest-fixture and energy generating vibratory device may be mounted to the wall of the unit within the operational constraints of the robotic arm.

Aspects

A variety of aspects of systems and apparatus have been described herein. A summary of a few select examples of such system and apparatus are provided below.

A 1st aspect is a cell culture system comprising: at least one cell culture vessel configured to culture cells using a plurality of parallel cell culture surfaces, wherein the at least one cell culture vessel comprises at least one port configured to allow material to flow into and out of the at least one cell culture vessel; manipulation apparatus configured to rotate the at least one cell culture vessel about a first rotation axis and about a second rotation axis, wherein the first rotation axis is perpendicular to the second rotation axis, wherein each of the first rotation axis and the second rotation axis are parallel a ground surface; pumping apparatus fluidly coupled to the at least one port of the at least one cell culture vessel and configured to pump material into and out of the at least one cell culture vessel through the at least one port; monitoring apparatus configured to monitor one or more parameters of the at least one cell culture vessel, the manipulation apparatus, and the pumping apparatus; and control apparatus operably coupled to the manipulation apparatus, the pumping apparatus, and the monitoring apparatus, wherein the control apparatus is configured to coordinate movement of the at least one cell culture vessel using the manipulation apparatus with the pumping of material into and out of the at least one culture vessel using the pumping apparatus.

A 2nd aspect is a system of 1st aspect, wherein the control apparatus is further configured to: move, using the manipulation apparatus, the at least one cell culture vessel into at least one fill position for filling the at least one cell culture vessel; and pump, using the pumping apparatus, cell culture medium into the at least one cell culture vessel after the manipulation apparatus has moved the at least one cell culture vessel into the at least one fill position.

A 3rd aspect is a system of 2nd aspect, wherein the at least one cell culture vessel is rotated 90 degrees about the first axis and 10 degrees about the second axis when in one of the at least one fill position.

A 4th aspect is a system of any of aspects 1-3, wherein the control apparatus is further configured to: move, using the manipulation apparatus, the at least one cell culture vessel into at least one emptying position for emptying the at least one cell culture vessel; and pump, using the pumping apparatus, cell culture medium out of the at least one cell culture vessel after the manipulation apparatus has moved the at least one cell culture vessel into one of the at least one emptying position.

A 5th aspect is a system of any of aspects 1-4, wherein the at least one cell culture vessel is rotated 90 degrees about the first axis and 10 degrees about the second axis when in one emptying position of the one or more emptying positions.

A 6th aspect is a system of any of aspects 1-5, wherein the monitoring apparatus comprises one or more position sensors coupled to the manipulation apparatus proximate the at least one cell culture vessel, wherein the one or more positions sensors are configured to sense the position of the at least one cell culture vessel relative to the ground surface, wherein the control apparatus is further configured to monitor the position of the at least one cell culture vessel relative to the ground surface using the one or more position sensors of the monitoring apparatus.

A 7th aspect is a system of any of aspects 1-6, wherein the monitoring apparatus comprises at least one pressure sensor fluidly coupled to each of the at least one cell culture vessels to measure the pressure in each of the at least one cell culture vessel, wherein the control apparatus is further configured to monitor the pressure of each of the at least one cell culture vessel to determine if the at least one cell culture vessel is one of effectively filled, effectively emptied, and faulty.

An 8th aspect is a system of any of aspects 1-7, wherein the monitoring apparatus comprises at least one load sensor coupled to each of the at least one cell culture vessels to measure the mass of each of the at least one cell culture vessel, wherein the control apparatus is further configured to monitor the mass of each of the at least one cell culture vessel for use in coordinating the movement of the at least one cell culture vessel using the manipulation apparatus with the pumping of material into and out of the at least one culture vessel using the pumping apparatus.

A 9th aspect is a system of any of aspects 1-8, wherein the control apparatus is further configured to modify the rate at which material is pumped into and out of the at least one culture vessel using the pumping apparatus based on the one or more monitored parameters of the at least one cell culture vessel.

A 10th aspect is a system of any of aspects 1-9, wherein the control apparatus is configured to move the at least one cell culture vessel and pump materials into or out of the at least one cell culture vessel at the same time.

An 11th aspect is a cell culture system comprising: at least one cell culture vessel configured to culture cells using a plurality of parallel cell culture surfaces, wherein the at least one cell culture vessel comprises at least one port configured to allow material to flow into and out of the at least one cell culture vessel; manipulation apparatus configured to rotate the at least one cell culture vessel about a first rotation axis and about a second rotation axis, wherein the first rotation axis is perpendicular to the second rotation axis, wherein each of the first rotation axis and the second rotation axis are parallel a ground surface; cell release apparatus configured to release cells adhered to the plurality of parallel cell culture surfaces of the at least one cell culture vessel; monitoring apparatus configured to monitor one or more parameters of the at least one cell culture vessel and the manipulation apparatus; and control apparatus operably coupled to the manipulation apparatus, the cell release apparatus, and the monitoring apparatus, wherein the control apparatus is configured to execute a cell release process using the cell release apparatus to release at least a portion of a plurality of cells adhered to the plurality of parallel cell culture surfaces of the at least one cell culture vessel.

A 12th aspect is a system of the 11th aspect, wherein the cell release apparatus comprises a shaking apparatus configured to shake the at least one cell culture vessel at a frequency greater than or equal to about 0.1 kHz and less than or equal to about 20 kHz to release at least a portion of a plurality of cells adhered to the plurality of parallel culture surfaces of the at least one cell culture vessel.

A 13th aspect is a system of the 11th aspect, wherein the cell release apparatus comprises ultrasonic transducer apparatus configured to provide ultrasonic energy to the at least one cell culture vessel at a frequency greater than or equal to about 10 kHz and less than or equal to about 30 kHz to release at least a portion of a plurality of cells adhered to the plurality of parallel cell culture surfaces of the at least one cell culture vessel

A 14th aspect is a system of any of aspects 11-13, wherein the cell release apparatus is coupled to manipulation apparatus and configured to one of shake at least a portion of the manipulation apparatus and deliver ultrasonic energy to at least a portion of the manipulation apparatus.

A 15th aspect is a system of any of aspects 11-13, wherein, to execute a cell release process using the cell release apparatus, the control apparatus is configured to: move the at least one cell culture vessel, using the manipulation apparatus, into contact with the cell release apparatus; and initiate the cell release apparatus to release at least a portion of a plurality of cells adhered to the plurality of parallel cell culture surfaces of the at least one cell culture vessel.

A 16th aspect is a system of any of aspects 11-13, wherein the cell release apparatus is moveably coupled to the manipulation apparatus to move about the at least one cell culture vessel to one of shake the at least one cell culture vessel and deliver ultrasonic energy to the plurality of parallel cell culture surfaces of the at least one cell culture vessel, and wherein, to execute a cell release process using the cell release apparatus, the control apparatus is configured to: move the cell release apparatus about the at least one cell culture vessel; and initiate the cell release apparatus to release at least a portion of a plurality of cells adhered to the plurality of parallel cell culture surfaces of the at least one cell culture vessel.

A 17th aspect is a system of any of aspects 11-16, wherein the cell culture system further comprises pumping apparatus fluidly coupled to the at least one port of the at least one cell culture vessel and configured to pump material into and out of the at least one cell culture vessel through the at least one port, and wherein the control apparatus is operably coupled to the pumping apparatus and is configured to execute an emptying process using the pumping apparatus and the manipulation apparatus after executing the cell release process, wherein the emptying process comprises pumping, using the pumping apparatus, cell culture medium out of the at least one cell culture vessel after the manipulation apparatus has moved the at least one cell culture vessel into at least one emptying position.

An 18th aspect is a cell culture system comprising: at least one cell culture vessel configured to culture cells using a plurality of parallel cell culture surfaces, wherein the at least one cell culture vessel comprises at least one port configured to allow material to flow into and out of the at least one cell culture vessel; manipulation apparatus configured to rotate the at least one cell culture vessel about a first rotation axis and about a second rotation axis, wherein the first rotation axis is perpendicular to the second rotation axis, wherein each of the first rotation axis and the second rotation axis are parallel a ground surface; pumping apparatus fluidly coupled to the at least one port of the at least one cell culture vessel and configured to pump material into and out of the at least one cell culture vessel through the at least one port; monitoring apparatus configured to monitor one or more parameters of the at least one cell culture vessel, the manipulation apparatus, and the pumping apparatus; and control apparatus operably coupled to the manipulation apparatus, the pumping apparatus, and the monitoring apparatus, wherein the control apparatus is configured to: monitor, using the monitoring apparatus, one or more parameters of the at least one cell culture vessel, the manipulation apparatus, and the pumping apparatus, and adjust one or more parameters of the at least one cell culture vessel, the manipulation apparatus, and the pumping apparatus based on the monitored one or more parameters.

A 19th aspect is a system of the 18th aspect, wherein the cell culture system further comprises incubation apparatus configured to incubate the at least one cell culture vessel when located therein, wherein the monitoring apparatus comprises at least one temperature sensor configured to measure the temperature inside the incubation apparatus, and wherein the control apparatus is operably coupled to the incubation apparatus to control the incubation apparatus, wherein the control apparatus is configured to: monitor the temperature inside the incubation apparatus; and adjust the temperature inside the incubation apparatus based on the monitored temperature.

A 20th aspect is a system of any of aspects 18-19, wherein the monitoring apparatus further comprises at least one fill sensor configured to monitor the fill volume of the at least one cell culture vessel, and wherein the control apparatus is configured to: monitor the fill volume of the at least one cell culture vessel; and adjust the fill volume of the at least one cell culture vessel using the pumping apparatus based on the monitored fill volume.

A 21st aspect is a system of any of aspects 18-20, wherein the monitoring apparatus comprises at least one of an oxygen sensor configured to monitor oxygen concentration in the at least one cell culture vessel, a carbon dioxide sensor configured to monitor carbon dioxide concentration in the at least one cell culture vessel, a glucose sensor configured to monitor glucose in the at least one cell culture vessel, an ammonium sensor configured to monitor ammonium concentration in the at least one cell culture vessel, a pH sensor configured to monitor pH in the at least one cell culture vessel, and a lactate sensor configured to monitor lactate in the at least one cell culture vessel.

A 22nd aspect is a system of any of aspects 18-21, wherein the monitoring apparatus comprises at least one pressure sensor configured to measure the pressure in the at least one cell culture vessel, wherein the control apparatus is configured to: apply pressure to at least one cell culture vessel using the pumping apparatus; monitor the pressure the at least one cell culture vessel using the at least one pressure sensor; and determine the integrity of the at least one cell culture vessel based on the monitored pressure.

A 23rd aspect is a system of any of aspects 1-22, wherein the system further comprises one or more outrigger portions configurable between a stowed configuration and a deployed configuration, wherein the one or more outrigger portions are configured to support the manipulator portion on the ground surface when in the deployed configuration.

Thus, embodiments of CELL CULTURE SYSTEM are disclosed. One skilled in the art will appreciate that the cell culture apparatuses and methods described herein can be practiced with embodiments other than those disclosed. The disclosed embodiments are presented for purposes of illustration and not limitation.

What is claimed is:

1. A cell culture system comprising:
at least one cell culture vessel configured to culture cells using a plurality of parallel cell culture surfaces, wherein the at least one cell culture vessel comprises at least one port configured to allow material to flow into and out of the at least one cell culture vessel;
manipulation apparatus configured to rotate the at least one cell culture vessel about a first rotation axis and about a second rotation axis, wherein the first rotation axis is perpendicular to the second rotation axis, wherein each of the first rotation axis and the second rotation axis are parallel a ground surface;
pumping apparatus fluidly coupled to the at least one port of the at least one cell culture vessel and configured to pump material into and out of the at least one cell culture vessel through the at least one port;
monitoring apparatus configured to monitor one or more parameters of the at least one cell culture vessel, the manipulation apparatus, and the pumping apparatus; and
control apparatus operably coupled to the manipulation apparatus, the pumping apparatus, and the monitoring apparatus, wherein the control apparatus is configured to coordinate movement of the at least one cell culture vessel using the manipulation apparatus with the pumping of material into and out of the at least one culture vessel using the pumping apparatus.

2. The system of claim 1, wherein the control apparatus is further configured to:
move, using the manipulation apparatus, the at least one cell culture vessel into at least one fill position for filling the at least one cell culture vessel; and
pump, using the pumping apparatus, cell culture medium into the at least one cell culture vessel after the manipulation apparatus has moved the at least one cell culture vessel into the at least one fill position.

3. The system of claim 2, wherein the at least one cell culture vessel is rotated 90 degrees about the first axis and 10 degrees about the second axis when in one of the at least one fill position.

4. The system of claim 1, wherein the control apparatus is further configured to:
move, using the manipulation apparatus, the at least one cell culture vessel into at least one emptying position for emptying the at least one cell culture vessel; and
pump, using the pumping apparatus, cell culture medium out of the at least one cell culture vessel after the manipulation apparatus has moved the at least one cell culture vessel into one of the at least one emptying position.

5. The system of claim 4, wherein the at least one cell culture vessel is rotated 90 degrees about the first axis and 10 degrees about the second axis when in one emptying position of the one or more emptying positions.

6. The system of claim 1, wherein the monitoring apparatus comprises one or more position sensors configured to sense the position of the at least one cell culture vessel relative to the ground surface,
wherein the control apparatus is further configured to monitor the position of the at least one cell culture vessel relative to the ground surface using the one or more position sensors of the monitoring apparatus.

7. The system of claim 1, wherein the monitoring apparatus comprises at least one pressure sensor fluidly coupled to the at least one cell culture vessels to measure the pressure in the at least one cell culture vessel,
wherein the control apparatus is further configured to monitor the pressure of each of the at least one cell culture vessel to determine if the at least one cell culture vessel is one of effectively filled, effectively emptied, and faulty.

8. The system of claim 1, wherein the monitoring apparatus comprises at least one load sensor to measure the mass of each of the at least one cell culture vessel,
wherein the control apparatus is further configured to monitor the mass of each of the at least one cell culture vessel for use in coordinating the movement of the at least one cell culture vessel using the manipulation apparatus with the pumping of material into and out of the at least one culture vessel using the pumping apparatus.

9. The system of claim 1, wherein the control apparatus is further configured to modify the rate at which material is pumped into and out of the at least one culture vessel using the pumping apparatus based on the one or more monitored parameters of the at least one cell culture vessel.

10. The system of claim 1, wherein the control apparatus is configured to move the at least one cell culture vessel and pump materials into or out of the at least one cell culture vessel at the same time.

11. A cell culture system comprising:
at least one cell culture vessel configured to culture cells using a plurality of parallel cell culture surfaces, wherein the at least one cell culture vessel comprises at least one port configured to allow material to flow into and out of the at least one cell culture vessel;
manipulation apparatus configured to rotate the at least one cell culture vessel about a first rotation axis and about a second rotation axis, wherein the first rotation axis is perpendicular to the second rotation axis, wherein each of the first rotation axis and the second rotation axis are parallel a ground surface;
cell release apparatus configured to release cells adhered to the plurality of parallel cell culture surfaces of the at least one cell culture vessel;
monitoring apparatus configured to monitor one or more parameters of the at least one cell culture vessel and the manipulation apparatus; and
control apparatus operably coupled to the manipulation apparatus, the cell release apparatus, and the monitoring apparatus, wherein the control apparatus is configured to execute a cell release process using the cell release apparatus to release at least a portion of a plurality of cells adhered to the plurality of parallel cell culture surfaces of the at least one cell culture vessel.

12. The system of claim 11, wherein the cell release apparatus comprises a shaking apparatus configured to shake the at least one cell culture vessel at a frequency greater than or equal to about 0.1 kHz and less than or equal to about 20 kHz to release at least a portion of a plurality of cells adhered to the plurality of parallel culture surfaces of the at least one cell culture vessel.

13. The system of claim 11, wherein the cell release apparatus comprises ultrasonic transducer apparatus configured to provide ultrasonic energy to the at least one cell culture vessel at a frequency greater than or equal to about 10 kHz and less than or equal to about 30 kHz to release at least a portion of a plurality of cells adhered to the plurality of parallel cell culture surfaces of the at least one cell culture vessel

14. The system of claim 13, wherein the cell release apparatus is coupled to manipulation apparatus and configured for one of shaking at least a portion of the manipulation apparatus and delivering ultrasonic energy to at least a portion of the manipulation apparatus.

15. Currently amended) The system of claim 13, wherein, to execute a cell release process using the cell release apparatus, the control apparatus is configured to:
move the at least one cell culture vessel, using the manipulation apparatus, into contact with the cell release apparatus; and
initiate the cell release apparatus to release at least a portion of a plurality of cells adhered to the plurality of parallel cell culture surfaces of the at least one cell culture vessel.

16. The system of claim 11, wherein the cell release apparatus is moveably coupled to the manipulation apparatus to move about the at least one cell culture vessel for one of shaking the at least one cell culture vessel and delivering ultrasonic energy to the plurality of parallel cell culture surfaces of the at least one cell culture vessel, and
wherein, to execute a cell release process using the cell release apparatus, the control apparatus is configured to:
move the cell release apparatus about the at least one cell culture vessel; and
initiate the cell release apparatus to release at least a portion of a plurality of cells adhered to the plurality of parallel cell culture surfaces of the at least one cell culture vessel.

17. The system of claim 11, wherein the cell culture system further comprises pumping apparatus fluidly coupled to the at least one port of the at least one cell culture vessel and configured to pump material into and out of the at least one cell culture vessel through the at least one port, and
wherein the control apparatus is operably coupled to the pumping apparatus and is configured to execute an emptying process using the pumping apparatus and the manipulation apparatus after executing the cell release process, wherein the emptying process comprises pumping, using the pumping apparatus, cell culture medium out of the at least one cell culture vessel after the manipulation apparatus has moved the at least one cell culture vessel into at least one emptying position.

18. A cell culture system comprising:
at least one cell culture vessel configured to culture cells using a plurality of parallel cell culture surfaces, wherein the at least one cell culture vessel comprises at least one port configured to allow material to flow into and out of the at least one cell culture vessel;
manipulation apparatus configured to rotate the at least one cell culture vessel about a first rotation axis and about a second rotation axis, wherein the first rotation axis is perpendicular to the second rotation axis, wherein each of the first rotation axis and the second rotation axis are parallel a ground surface;
pumping apparatus fluidly coupled to the at least one port of the at least one cell culture vessel and configured to pump material into and out of the at least one cell culture vessel through the at least one port;
monitoring apparatus configured to monitor one or more parameters of the at least one cell culture vessel, the manipulation apparatus, and the pumping apparatus; and
control apparatus operably coupled to the manipulation apparatus, the pumping apparatus, and the monitoring apparatus, wherein the control apparatus is configured to:
monitor, using the monitoring apparatus, one or more parameters of the at least one cell culture vessel, the manipulation apparatus, and the pumping apparatus, and
adjust one or more parameters of the at least one cell culture vessel, the manipulation apparatus, and the pumping apparatus based on the monitored one or more parameters.

19. The system of claim 18, wherein the cell culture system further comprises incubation apparatus configured to incubate the at least one cell culture vessel when located therein, wherein the monitoring apparatus comprises at least one temperature sensor configured to measure the temperature inside the incubation apparatus, and
wherein the control apparatus is operably coupled to the incubation apparatus to control the incubation apparatus, wherein the control apparatus is configured to:
monitor the temperature inside the incubation apparatus; and
adjust the temperature inside the incubation apparatus based on the monitored temperature.

20. The system of claim 18, wherein the monitoring apparatus further comprises at least one fill sensor configured to monitor the fill volume of the at least one cell culture vessel, and
wherein the control apparatus is configured to:
monitor the fill volume of the at least one cell culture vessel; and
adjust the fill volume of the at least one cell culture vessel using the pumping apparatus based on the monitored fill volume.

21. The system of claim 18, wherein the monitoring apparatus comprises at least one of an oxygen sensor configured to monitor oxygen concentration in the at least one cell culture vessel, a carbon dioxide sensor configured to monitor carbon dioxide concentration in the at least one cell culture vessel, a glucose sensor configured to monitor glucose in the at least one cell culture vessel, an ammonium sensor configured to monitor ammonium concentration in the at least one cell culture vessel, a pH sensor configured to monitor pH in the at least one cell culture vessel, and a lactate sensor configured to monitor lactate in the at least one cell culture vessel.

22. The system of claim 18, wherein the monitoring apparatus comprises at least one pressure sensor configured to measure the pressure in the at least one cell culture vessel, wherein the control apparatus is configured to:
apply pressure to at least one cell culture vessel using the pumping apparatus;
monitor the pressure the at least one cell culture vessel using the at least one pressure sensor; and
determine the integrity of the at least one cell culture vessel based on the monitored pressure.

23. The system of claim 1, wherein the system further comprises one or more outrigger portions configurable between a stowed configuration and a deployed configuration, wherein the one or more outrigger portions are configured to support the manipulator portion on the ground surface when in the deployed configuration.

24. The system of claim 11, wherein the system further comprises one or more outrigger portions configurable between a stowed configuration and a deployed configuration, wherein the one or more outrigger portions are configured to support the manipulator portion on the ground surface when in the deployed configuration.

25. The system of claim 18, wherein the system further comprises one or more outrigger portions configurable between a stowed configuration and a deployed configuration, wherein the one or more outrigger portions are configured to support the manipulator portion on the ground surface when in the deployed configuration.
